(19) United States
(12) Patent Application Publication
(10) Pub. No.: US 2011/0046039 A1
(43) Pub. Date: Feb. 24, 2011

(54) POST-EXPOSURE PROPHYLAXIS AND TREATMENT OF INFECTIONS

(75) Inventors: Serguei G. Popov, Bristow, VA (US); Taissia Popov, Bristow, VA (US); Virginia Espina, Rockville, MD (US); Charles Bailey, Cross Junction, VA (US); Lance A. Liotta, Bethesda, MD (US); Emanuel Petricoin, Gainesville, VA (US)

Correspondence Address: MILLEN, WHITE, ZELANO & BRANIGAN, P.C., 2200 CLARENDON BLVD., SUITE 1400, ARLINGTON, VA, 22201, US

(73) Assignee: George Mason Intellectual Properties Inc., Fairfax, VA (US)

(21) Appl. No.: 12/530,538

(22) PCT Filed: Mar. 6, 2008
(86) PCT No.: PCT/US08/56038

§ 371 Date: Mar. 8, 2010

Related U.S. Application Data
(60) Provisional application No. 60/905,916, filed on Mar. 9, 2007.

(51) Int. Cl. A61K 38/07; A61P 31/04

(52) U.S. Cl. 514/2.4

(57) ABSTRACT

The invention provides methods and materials for identifying agents for preventing and/or treating anthrax and similar diseases. Embodiments provide strains and model systems for studying non-lethal and lethal exposure to anthrax and similar disease vectors. Embodiments provide materials and methods for using the strains and model systems for differential profiling, such as proteomic profiling, such as differentiation phosphorylation profiling, to target identification and therapeutics discovery and development. Embodiments provide pharmaceutically acceptable compositions, and methods for using them to prevent and/or treat anthrax and similar diseases comprising an agent that decreases the activity of caspase ¼, such as YVAD, and/or an agent that increases the phosphorylation of AKT, such as IB-MECA or Cl-IB-MECA, together with, in particular embodiments, an antibiotic, such as ciprofloxacin. Kits comprising the same are provided as well, among other things.

### REFERENCE TO RELATED APPLICATION

This application is a continuation-in-part of U.S. provisional application No. 60/905,916, filed on 9 Mar. 2007, of which priority is claimed and which is herein incorporated by reference in its entirety.

### STATEMENT REGARDING GOVERNMENT FUNDING

The work resulting in the subject matter herein described was funded in part by US Army Project Number DAMD17-03-C-0122.

### FIELD OF THE INVENTION

The invention relates to preventing and treating exposure to and or infection by anthrax and other microbes. It relates as well to developing methods and materials therefor, and to model systems for studying and for developing the same.

### BACKGROUND OF THE INVENTION

Effective prophylaxes and treatment of infectious diseases remain a challenging task, despite tremendous advances in antibiotic and chemo therapies during the last century. One reason the challenge is on-going is that the infectious disease agents are mutable. Ever evolving microbial vectors of diseases effectively adapt to new drugs, giving rise to previously unknown strains and variants that are drug resistant. The emergence of drug-resistance in microbial disease vectors not only limits the choice of effective therapeutics but also increases the need for timely and accurate diagnoses. There is thus an on-going and in some instances an increasingly acute need for better methods and therapeutic agents for preventing and treating diseases caused by microbial vectors.

The need for new and better approaches to combating infections, moreover, is not limited to disease vectors that arise naturally. It is, in fact, especially evident in the area of biodefense, which must address the threat of biological weapon attacks with highly virulent agents, which may have been additionally engineered to resist existing vaccines and therapeutics.

Bacillus anthracis, the causative agent of anthrax, is one example of a biowarfare threat. Inhalation of anthrax spores causes a severe infection. Historically, 92% of people exposed to anthrax by inhalation die, regardless of treatment.¹ In the relatively recent case of inhalation anthrax exposure in the US in 2001 the mortality rate was 55%, an unacceptably high rate that would spell disaster in the event of a large-scale attack. The mortality rate likely would be even higher for antibiotic and/or vaccine-resistant recombinant variants of B. anthracis, which have been reported.^{2,3}

Currently approved drugs for treating inhalation anthrax treatment are limited to the fluoroquinolone and tetracycline classes of antibiotics. These drugs, unfortunately, are not effective against the septic shock typically engendered by B. anthracis infection, and they do not prevent or ameliorate its consequences: hypoxic organ failure and circulatory collapse.⁴

Although the events leading to death of infected animals and patients remain incompletely understood, the virulence of B. anthracis can be attributed mainly to the lethal toxin (“LeTx”) and the edema toxin (“EdTx”), encoded by the pXO1 plasmid, and to the anti-phagocytic capsule encoded by the pXO2 plasmid.⁵

Much effort has been devoted to developing agents that specifically inhibit LeTx proteolytic activity. Only recently, however, have promising results of this approach been reported in an animal model of anthrax.⁶ Similar approaches targeting other anthrax proteolytic factors have demonstrated post-exposure protection in mice.⁷ Despite the promising results, it is not yet clear that this approach will provide much needed improvements in treatment efficacy.

There is therefore an urgent need for improved therapeutics and therapeutic methods for treating anthrax and other infectious diseases, and for methods for their development.

### SUMMARY

It is therefore among the aspects, objects, and embodiments of the invention herein disclosed to provide, among other things, model systems for studying infectious processes and aspects of diseases such as anthrax, materials and methods for studying the same, and for identifying therapeutic targets thereby, materials and methods for preventing and/or treating anthrax and like diseases, and among other things, kits comprising the same.

The following numbered paragraphs are illuminative of various aspects and embodiments of the invention herein; but, taken alone or in any combination they are in no way exhaustive or otherwise limitative of the invention in all its many and diverse aspects, objects, embodiments, and the like. The phrase used below, “any of the foregoing or the following” refers to any of the foregoing or the following numbered paragraphs. The subject matter is set out in the paragraphs in this manner to indicate that some or all of the subject matter of any one or more paragraphs may be combined with some or all of the subject matter of any one or more other paragraphs. The paragraphs and the subject matter therein described are thus provided by way of clear written description support for claims directed to any such subject matter as may be delimited by reciting subject matter as set forth in any combination of part or of all of any one, two, or more of these numbered paragraphs.

A1. A method for preventing and/or treating an anthrax infection, comprising administering to a subject at risk for or suffering from an anthrax infection an agent that decreases the activity of caspase ¼, wherein said agent is administered in an effective amount and by an effective route for preventing and/or treating said anthrax infection.

A2. A method according to any of the foregoing or the following, wherein the agent is YVAD.

A3. A method according to any of the foregoing or the following, further comprising administering an antibiotic to said subject, wherein said antibiotic is administered in an amount and by a route effective for preventing and/or treating said anthrax infection in combination with said agent.

A4. A method according to any of the foregoing or the following, wherein the antibiotic is ciprofloxacin.

B 1. A method for preventing and/or treating anthrax infections, comprising administering to a subject at risk for or suffering from an anthrax infection an agent that increases the phosphorylation of AKT, wherein said agent is administered in an effective amount and by an effective route for preventing and/or treating said anthrax infection.

B2. A method according to any of the foregoing or the following, wherein the agent is an agonist of an adenosine A3 receptor.

B3. A method according to any of the foregoing or the following, wherein the agonist is IB-MECA or Cl-IB-MECA.

B4. A method according to any of the foregoing or the following, further comprising administering an antibiotic to said subject, wherein said antibiotic is administered in an amount and by a route effective for preventing and/or treating said anthrax infection in combination with said agent.

B5. A method according to any of the foregoing or the following, wherein the antibiotic is ciprofloxacin.

C1. A method for preventing and/or treating anthrax infections, comprising administering to a subject at risk for or suffering from anthrax infection a first agent that inhibits the activity of caspase ¼ and a second agent that increases the phosphorylation of AKT, wherein said first and said second agents each are administered in an amount and by a route effective for preventing and/or treating said anthrax infection in combination with one another.

C2. A method according to any of the foregoing or the following, wherein the agent that increases the phosphorylation of AKT is an agonist of an adenosine A3 receptor.

C3. A method according to any of the foregoing or the following, wherein the agent that increases the phosphorylation of AKT is IB-MEGA or Cl-IB-MECA.

C4. A method according to any of the foregoing or the following, wherein the agent that inhibits the activity of caspase ¼ is YVAD.

C5. A method according to any of the foregoing or the following, wherein the agent that increases the phosphorylation of AKT is an agonist of an adenosine A3 receptor.

C6. A method according to any of the foregoing or the following, wherein the agent that increases the phosphorylation of AKT is 1B-MECA or Cl-IB-MECA.

C7. A method according to any of claims of the foregoing or the following, further comprising administering an antibiotic to said subject, wherein said antibiotic is administered in an amount and by a route effective for preventing and/or treating said anthrax infection in combination with said agents.

C8. A method according to any of the foregoing or the following, wherein the antibiotic is ciprofloxacin.

D1. A pharmaceutically acceptable composition comprising an agent that decreases the activity of caspase ¼.

D2. A pharmaceutically acceptable composition according to any of the foregoing or the following, wherein the agent is YVAD.

D3. A pharmaceutically acceptable composition, comprising an agent that increases the phosphorylation of AKT.

D4. A pharmaceutically acceptable composition according to any of the foregoing or the following, wherein the agent is an agonist of an adenosine A3 receptor.

D5. A pharmaceutically acceptable composition according to any of the foregoing or the following, wherein the agent is IB-MEGA or Cl-IB-MECA.

D6. A pharmaceutically acceptable composition comprising a first agent that decreases the activity of caspase ¼ and a second agent that increases the phosphorylation of AKT.

D7. A pharmaceutically acceptable composition according to any of the foregoing or the following, wherein the first agent is YVAD.

D8. A pharmaceutically acceptable composition according to any of the foregoing or the following, wherein said second agent is an agonist of an adenosine A3 receptor.

D9. A pharmaceutically acceptable composition according to any of the foregoing or the following, wherein the second agent is IB-MECA or Cl-IB-MECA.

D10. A pharmaceutically acceptable composition according to any of the foregoing or the following, wherein the second agent is an agonist of an adenosine A3 receptor.

D11. A pharmaceutically acceptable composition according to any of the foregoing or the following, wherein the second agent is IB-MECA or Cl-IB-MECA.

D12. A pharmaceutically acceptable composition according to any of the foregoing or the following, further comprising an antibiotic.

D13. A pharmaceutically acceptable composition according to any of the foregoing or the following, wherein the antibiotic is ciprofloxacin.

D14. A pharmaceutically acceptable composition according to any of the foregoing or the following, wherein the composition is effective for preventing and/or treating anthrax infection.

E1. A kit, comprising in one or more containers a pharmaceutically acceptable composition comprising an agent that decreases the activity of caspase ¼ and instructions for the pharmaceutical use thereof.

E2. A kit according to according to any of the foregoing or the following, wherein the agent is YVAD.

E3. A kit, comprising in one or more containers a pharmaceutically acceptable composition comprising an agent that increases the phosphorylation of AKT and instructions for the pharmaceutical use thereof.

E4. A kit according to any of the foregoing or the following, wherein the agent is an agonist of an adenosine A3 receptor.

E5. A kit according to any of the foregoing or the following, wherein the agent is IB-MECA or Cl-IB-MECA.

E6. A kit, comprising in one or more containers a pharmaceutically acceptable composition comprising a first agent that decreases the activity of caspase ¼, a second agent that increases the phosphorylation of AKT, and instructions for the pharmaceutical use thereof.

E7. A kit according to any of the foregoing or the following, wherein said first agent is YVAD.

E8. A kit according to any of the foregoing or the following, wherein said second agent is an agonist of an adenosine A3 receptor.

E9. A kit according to any of the foregoing or the following, wherein said second agent is IB-MECA or Cl-IB-MECA.

E10. A kit according to any of the foregoing or the following, further comprising an antibiotic.

E11. A kit according to any of the foregoing or the following, wherein the antibiotic is ciprofloxacin.

F1. A method for identifying pathogenic host responses engendered by virulence factors encoded by the anthrax pXO1 plasmid, comprising:
(A) exposing a first plurality of cells to a pathogenic first Bacillus anthracis Sterne pXO1⁺, pXO2⁻ strain of bacteria, and determining at least one physiological response of said cells resulting from said exposure;
(B) exposing a second plurality of said cells to a non-pathogenic second Bacillus anthracis Sterne pXO1⁻, pXO2⁻ strain isogenic to said first strain, and determining said at least one physiological response of said cells resulting from said exposure; and
(C) identifying said pathological host responses to virulent factors encoded by said pXO1 plasmid by comparing said determination of (A) with that of (B).

F2. A method according to any of the foregoing or the following, wherein the cells are human small airway epithelial cells.

G1. A pair of Bacillus anthracis strains, wherein the pair of strains are isogenic and matched except that one strain of said pair is pathogenic, and the other strain of said pair is not pathogenic.

G2. A pair of Bacillus anthracis strains, wherein the pair of strains are isogenic, except that one strain of said pair is pXO1⁺, pXO2⁻ and the other strain is pXO1⁻, pXO2⁻.

G3. Two types of Bacillus anthracis strain Sterne bacterium, wherein the two types are the same except that one is pXO1⁺, pXO2⁻ and the other is pXO1⁻, pXO2⁻.

H1. A method for studying the effects of anthrax exposure and/or infection, comprising growing human small airway epithelial cells in vitro and exposing the cells to germinating anthrax spores.

H2. A method according to any of the foregoing or the following, wherein the anthrax is early stage anthrax.

H3. A method according to any of the foregoing or the following, wherein the effects on the lung epithelial cells of exposure to the germinating anthrax spores is studied by comparing an aspect of (a) an experimental group of cells exposed to germinating anthrax spores to (b) a control group of cells exposed to the same conditions without exposure to viable spores, wherein the experimental and control cells otherwise are the same cells and are treated in the same way.

H4. A method according to any of the foregoing or the following, wherein the effects of exposure to germinating anthrax spores is assessed by following a time course of changes brought about by said exposure.

H5. A method according to any of the foregoing or the following, wherein the effects of exposure to germinating anthrax spores is assessed by determining differences in any one or more of the following: gene expression, protein expression, and protein modification.

H6. A method according to any of the foregoing or the following, wherein the effects of exposure to germinating anthrax spores is assessed by determining differences in protein phosphorylation in exposed and unexposed cells.

H7. A method according to any of the foregoing or the following, wherein the effects of exposure to germinating anthrax spores is assessed by determining differences in protein phosphorylation of a panel of proteins, wherein the number of proteins in the panel is any of 10-100, 25-250, 50-500, 100-1,000, 500-5,000, 1,000-10,000, 2,000-20,000, or 5,000-25,0000 or more.

H8. A method according to any of the foregoing or the following, wherein the effects of exposure to germinating anthrax spores is assessed by determining differences in amounts of proteins in a panel of proteins, wherein the number of proteins in the panel is any of 10-100, 25-250, 50-500, 100 1,000, 500 5,000, 1,000 10,000, 2,000-20,000, or 5,000-25,0000 or more.

H9. A method according to any of the foregoing or the following, wherein the effects of exposure to germinating anthrax spores is assessed by determining differences in mRNAs in the control and experimental cells.

H10. A method according to any of the foregoing or the following, wherein the effects of exposure to germinating anthrax spores is assessed by determining differences in a population of mRNAs, wherein the number of mRNAs in the population is 10-100, 25-250, 50-500, 100-1,000, 500-5,000, 1,000-10,000, 2,000-20,000, or 5,000-25,0000 or more.

### Glossary

“A3AR” means adenosine A3 receptor.

“A3ARs” means adenosine A3 receptors.

“AKT” means a serine/threonine protein kinase that is also referred to in the literature as Akt, Akt/PKB, PKB, and protein kinase B. AKT is the cellular homologue of the viral oncogene v-Akt. The three AKT isoforms that have been identified in mammals, thus far, generally are referred to as, respectively: (a) Akt1, AKT1, PKBa/Akt1, and PKBa; (b) Akt2, AKT2, PKBb/Akt2, and PKB2, and (c) Akt3, AKT3, PKBg/Akt3, and PKBg. The viral oncogene, v-Akt, and the human AKT1 and AKT2 genes were first described in Staal, S. P., Proc Natl Acad Sci USA. 84(14): 5034-7 (1987), which is herein incorporated by reference in its entirety, particularly as to AKT proteins, their structure, and functions.

“AKT ½” means AKT1 and/or AKT2 each as described for “AKT” above.

“cAMP” means cyclic AMP (i.e., cyclic adenosine monophosphate).

“Cl-IB-MECA” means Cl substituted IB-MECA, that is: 2-chloro-N⁶-(3-iodobenzyl)-adenosine-5′-N-methyluronamide.

“EdTx” means edema toxin of Bacillus anthracis.

“ERK” means extracellular signal-regulated mitogen activated protein kinase.

“ERK ½” means “ERK” (as defined above) isoforms 1 and 2.

“GSK” means glycogen synthase kinase.

“GSK3” means glycogen synthase kinase 3.

“HSAECs” means human small airway epithelial cells.

“IB-MECA” means N⁶-(3-iodobenzyl)-adenosine-5′-N-methyluronamide.

“LeTx” means lethal toxin of Bacillus anthracis.

“MAPKs” means mitogen activated protein kinases.

“Non-pathogenic conditions” means not disease causing.

“Pathogenic conditions” means disease causing.

“Pharmaceutical use” means use for the prevention or treatment of a disorder or disease or the like, or of its effects, side effects, symptoms, or sequelae, inter alia, particularly, but not exclusively in humans.

“Pharmaceutically acceptable” means acceptable for pharmaceutical use, such as but not exclusively limited to, compositions and uses approved for medical use by the US FDA or by a counterpart agency charged with granting such approval in venues outside the US.

“Pharmaceutically acceptable composition” means a composition that is pharmaceutically acceptable.

“Pharmaceutically effective” means effective for a pharmaceutical use, achieving a desired prophylactic or therapeutic effect.

“Post-translational protein modification” means modifications of proteins that occur after cellular polypeptide synthesis has occurred. Many post-translational modifications of proteins are known that occur naturally. Often these modifications have significant roles in controlling protein transport, compartmentalization, interaction with other cell components, activity, and physiological properties, such as persistence and clearance, to name just a few. Among the more commonly occurring post-translational modifications are glycosylation, phosphorylation, methylation, acetylation, ubiquitinylation, and ADP-ribosylation (to name just a few).

“Prevent” means to block from occurring.

“Post-exposure” means after exposure.

“Prophylaxis” means to protect against, at best to prevent.

“System wide” denotes a plurality comprising some—but by no means necessarily all—elements of a class of elements in a system. For instance, a system wide analysis of signaling proteins means, as used herein, an analysis of a sampling (which may be random or selective) of signaling proteins in a system, which need not, but may, include all signaling proteins,

“Treat” means to administer so as to ameliorate, retard, stop, reverse, or cure a disorder or disease or the like, or its effects, side effects, symptoms, or sequelae, inter alia.

“YVAD” means acetyl-tyrosyl-valyl-alanyl-aspartyl-chloromethylketone.

“z-VAD” means z-Val-Ala-Asp(OMe)-fluoromethylketone.

### BRIEF DESCRIPTION OF THE FIGURES

FIG. 1 is a chart that shows signaling protein phosphorylation in HSAECs exposed to (i) anthrax spores of the non-pathogenic delta Sterne strain (upper row of each protein panel), or (ii) anthrax spores of the toxigenic Sterne strain (lower row), at MOIs of 1 and 10. Phosphorylation was detected using a panel of signaling protein-specific phosphorylation sensitive antibodies. Signaling proteins thus determined are indicated on the right side of the chart. All results are normalized to untreated (control) cells. No change from the control is indicated by black. Boxes without dashes show increases. Boxes with dashes show decreases. Degrees of increase or decrease are indicated by grey scale.

FIG. 2 is a graph showing that EdTx modulates AKT phoshphorylation in HSAECs in a time- and concentration-dependent manner. The cAMP inducer Foskolin, served as a positive control.

FIG. 3 is a set of charts showing survival of DBA/2 mice after infection and treatment with various agents and combinations of agents as follows:

FIG. 3A: ciprofloxacin (50 mg/kg) or YVAD (2.5 mg/kg) alone, and in combination.

FIG. 3B: ciprofloxacin (50 mg/kg) or YVAD (12.5 mg/kg) alone, and in combination.

FIG. 3C: Cl-IB-MECA at 0.05 mg/kg, 0.15 mg/kg, and 0.3 mg/kg.

FIG. 3D: ciprofloxacin at 50 mg/kg alone, and in combination with Cl-IB-MECA at 0.05 mg/kg, 0.15 mg/kg, and 0.3 mg/kg.

FIG. 3E: ciprofloxacin at 50 mg/kg alone, a combination of Cl-IB-MECA at 0.15 mg/kg and YVAD at 2.5 mg/kg, and a combination of Cl-IB-MECA at 0.15 mg/kg, ciprofloxacin at 50 mg/kg, and YVAD at 2.5 mg/kg.

FIG. 3F: ciprofloxacin at 50 mg/kg alone, a combination of Cl-IB-MECA at 0.3 mg/kg and YVAD at 12.5 mg/kg, and a combination of Cl-IB-MECA at 0.3 mg/kg, ciprofloxacin at 50 mg/kg, and YVAD at 12.5 mg/kg.

### DESCRIPTION OF THE INVENTION

Preventing and treating diseases, such as anthrax, that cannot be adequately prevented or treated with currently available therapeutics will require not only new therapeutics that target the infectious agent but also new therapeutics that eliminate or mitigate pathogenic host responses to infection. The present invention provides, in embodiments, methods for the identification of novel therapeutic targets and novel therapies.

The invention provides highly effective post-exposure agents and treatment strategies for preventing and/or treating microbial infections and diseases that target one or more host responses, rather than the infectious organism. In embodiments the treatments are mediated through specific pro-survival pathways. In embodiments the microbial infection is anthrax and the microbe is Bacillus anthraces.

In embodiments the therapeutic agents and methods have no direct anti-microbial effect and/or no direct effect on the action of microbial toxins. In embodiments the agents are one or more of a modulator of host cell inflammatory response and/or a mediator of host cell apoptopic response. In embodiments the agents are one or more of a caspase inhibitor and/or a A3AR agonist.⁴⁴

In embodiments the agents and/or therapies have a synergistic effect on post-exposure survival in combination with one or more anti-microbial agents. In embodiments in this regard the antibiotic dose is low. In embodiments the antibiotic is a member of the ciprofloxacin class or the tetracycline class of antibiotics. In embodiments the antibiotic is ciprofloxacin.

Embodiments of the invention provide systems biology methods for identifying novel therapeutic targets, novel therapeutics, and novel therapies. In embodiments the methods comprise system wide analysis of proteins under non-pathogenic and pathogenic conditions. In embodiments the methods comprise system wide analysis of signaling proteins under pathogenic and non-pathogenic conditions. In embodiments the methods comprise system wide analysis of post-translation modification of proteins under pathogenic and non-pathogenic conditions. In embodiments the methods comprise system wide analysis of phosphorylation of proteins under pathogenic and non-pathogenic conditions. In embodiments the methods comprise system wide analysis of post-translational modification of signaling proteins under pathogenic and non-pathogenic conditions. In embodiments the methods comprise system wide analysis of phosphorylation of signaling proteins under pathogenic and non-pathogenic conditions.

In embodiments the methods comprise using arrays for system wide analysis. In embodiments the arrays comprise a plurality of antibodies. In embodiments the antibodies are specific for a corresponding plurality of proteins. In embodiments the antibodies are specific for a plurality of proteins. In embodiments the antibodies are specific for a corresponding plurality of modifications of a corresponding multiplicity of proteins. In embodiments the antibodies are specific for post-translational modifications of signaling proteins. In embodiments the antibodies are specific for specific phosphorylations of specific signaling proteins.

In embodiments the methods comprise analysis of host responses in host cells exposed to and/or infected with either one of a matched pair of isogenic strains of a disease vector, wherein one member of the pair is pathogenic and the other member is not pathogenic.

In embodiments the disease vector is Bacillus anthracis. In embodiments the disease is anthrax.

In embodiments the host cells are small airway epithelial cells. In embodiments the host cells are human small airway epithelial cells.

Phosphorylation and Signaling Pathways in Anthrax Infection

As an example of an embodiment of the invention for determining potential novel therapeutic targets, the impact of anthrax infection on host signaling pathways was studied using cell culture conditions that mimic a human exposure route. In the case of inhalation anthrax, the outcome of the spore interaction with the epithelial surface of the lungs has long been recognized as one of the factors contributing to bacterial virulence. Persistence of the dormant spores in the lungs remains a challenging problem in the current antibiotic-based prophylaxis of anthrax,⁸ as exposed persons are required to take antibiotics for at least 60 days (See Inglesby et al., Anthrax as a biological weapon 2002: updated recommendations for management, JAMA 287(17):2236-52 (2002), which is herein incorporated by reference in its entirety, particularly in points pertinent to the foregoing.

In a novel aspect of the invention, human lung epithelial cells were used as an in vitro model of early inhalation exposure, and cell signaling events were monitored before and during exposure to the germinating anthrax spores and vegetative cells. In addition, a novel protein microarray platform was employed for multiplexed analysis of phosphorylation-driven cell signaling cascades, as recently described for tissue-based cancer studies.⁹ Using this technology, which takes advantage of the large and growing number of antibodies that recognize proteins only when they are phosphorylated, it is possible to quantitatively and sensitively measure over 100 kinase substrates from a few thousand cells.

Matched Isogenic Pathogenic and Non-Pathogenic Anthrax for Model Studies

To more specifically identify cell signaling pathways that are causally important/related to the infectious process directly, the signal pathway profiling was performed using a toxigenic anthrax strain Sterne (pXO1⁺, pXO2⁻) and compared to the impact of bacterial exposure on lung epithelial host cell signaling with the isogenic, non-pathogenic anthrax strain (delta-Sterne (pXO1⁻, pXO2⁻) profiled in the same manner. The pathogenic and non-pathogenic strains provided herein are a means to identify the pathogenic host responses due to the expression of anthrax virulence factors encoded by the pXO1 plasmid, and represent an important advance over previous studies that failed to utilize isogenic matched strains and typically report results using only virulent strains or toxins.^{10,11,12,13,14} The matched isogenic approach also provides the opportunity to study the late bacteremic stages of infection, when anthrax-encoded secreted toxins along with other pathogenic factors are thought to be involved in the damage to the host vital organs with high epithelial content, such as lung, liver, spleen, and kidney. Therefore data collected and results generated by the exposure of primary lung epithelial cells to a pathogenic and non-pathogenic strain provide general guidance regarding therapeutic mitigation of anthrax, and validate the biological significance of findings in animal models systems (e.g., spore challenged mice), as described herein.

The cultured lung epithelial cells challenged with anthrax spores serve as sensors of infection, and are sensitive to pathogenic factors encoded by the toxigenic plasmid pXO1. The ability to broadly measure the activation and phosphorylation of cell signaling pathways using a novel proteomic assay provides critical information about which specific signaling networks, out of the myriad of potential candidates, are altered. The use of isogenic matched non-pathogenic and pathogenic strains for host challenge studies also is highly valuable for the facile determination of specifically affected networks. Ultimately, the information gleaned from the in vitro cell line experiments provides a rationale for pharmacological approaches to use in animal models. The mouse studies described herein, for example, reveal that the pathway effects observed in cell culture are not simply correlative findings, but underpin the most important direct biological outcome of anthrax exposure: mortality.

Among these pathways, interference with the cell survival program through modulation of MAPK and AKT activation may represent an important part of anthrax pathogenic strategy. This is in agreement with the recent report on the inhibition of T lymphocyte AKT phosphorylation in vivo by LeTx,¹⁵ and allows rational explanation of some poorly understood pathophysiological features of anthrax, as discussed below.

Liver damage and cardiovascular collapse are considered to be the major causes of death of anthrax toxin-challenged animals See, for instance, Cui et al. Am. J. Physiol. Regul. Integr. Comp. Physiol. 286: R699-709 (2004) and Moayeri et al., Curr. Opin. Microbial. 7: 19-24 (2004), each of which is herein incorporated in its entirety in this regard. Lethality during cardiovascular anthrax lethal toxin infusion is associated with circulatory shock but not with inflammatory cytokine or nitric oxide release in rats. The results herein described demonstrate that enhanced GSK-3β phosphorylation at low MOI is followed by its down-regulation at higher number of bacteria. The results show further that EdTx through generation of cAMP is able to modulate AKT, and this suggests, based on the fact that GSK-3β is a specific substrate of AKT kinase activity, that the downstream activity of GSK-3β regulating glycogenolysis by liver and skeletal muscles may be responsible for the glucose level perturbation in anthrax.¹⁶

LeTx has also been shown to modify transcription of the GSK-3β-mediated genes in macrophages by an unknown mechanism.¹⁰ The physiological effects of cAMP on liver and other organs mimic stimulation of the vascular adrenergic receptors (ARs). The epinephrine-like activity of culture filtrates from B. anthracis, and other observations such as the “sudden death” phenomenon commonly observed in anthrax patients, implicate central nervous system (CNS) involvement in the action of anthrax toxins, and indicate the presence of a low-molecular-weight, endogenously produced AR agonist, which has not yet been identified.^{37,17} The results set forth herein indicate that the agonist is cAMP, produced under CNS control as a mediator of physiological stress and/or as a result of EdTx enzymatic activity.

Mediation of anthrax pathogenesis by the action of cAMP on the AKT signaling pathway explains, at least partly, the ability of the β-AR agonist isoproterenol to prevent the life threatening drop in blood pressure caused by intensive vasodilation in monkeys administered lethal doses of crude anthrax toxin.¹⁸ Elevation of cAMP quickly improves endothelial barrier function. However, prolonged generation of cAMP by EdTx produces the opposite effect, resulting in low blood oxygenation and, ultimately, to septic shock. As a result, therapies attempting to utilize cAMP to protect the vasculature barrier function have often failed.¹⁹ In addition, chronic exposure of cardiomyocytes to cAMP leads to hypercontracture and toxicity, an effect that mimics the effect of catecholamines, such as norepinephrine, acting through β1-AR.³⁶ Finally, LeTx inhibition of p38 signaling should block the anti-apoptotic effect of β2-AR stimulation acting through PI3 kinase and AKT.²⁰

### Examples

The invention is further described by way of the following examples. The examples are by way of illustration only and are not limitative. A full understanding of the invention requires reading the entirety of the application disclosure, including all of the foregoing and the following text (including the description, abstract, and claims) and the figures. Such an understanding furthermore requires (and assumes) that the same will be read, understood, and interpreted with the knowledge, understanding, and insight of a person skilled in the art(s) pertinent to the invention and necessary to its understanding and application.

### Example 1

### Reagents and Antibodies

Cell culture reagents were obtained from Cellgro (Herndon., Va.).

Antibodies against total and phosphorylated forms of the following proteins used for reverse phase protein microarray and Western blot analyses were obtained from Cell Signaling Technology (Beverly, Mass.).

Antibodies (identified by their specificities) were used at the following dilutions:

TABLE 1
1:20 for p70 S6 Kinase (Thr389)
1:50 for c-Abl (Thr735), Stat5 (Tyr694), and 4E-BP1 (Ser65)
1:100 for Akt (Ser473), MEK1/2 (Ser 217/221), pIKBa (Ser32/Ser36),
Bad (Ser112, 155 and 136), 4E-BP1 (Thr70), GSK-3α/β
(Ser21/9), CREB (Ser 133), Stat3 (Ser727, Tyr705), Jak1
(Tyr1022/1023), FAK (Tyr576/577), Etk (Tyr 40), Elk-1 (Ser383),
and MARCKS (Ser152/156)
1:200 for mTOR (Ser2448), eNOS (Ser1177), Pyk2 (Tyr402), FADD
(Ser194), Stat6 (Tyr641) and Bcl-2 Ser70
1:250 for p38 (Thr180/Tyr182), IL-1β-cleaved (Asp116); 1:400 for
p90RSK (Ser380); 1:500 for PKC-δ (Thr505), Src (Tyr 527),
PKC-α/β (Thr638/641), PKC-θ (Thr538), caspase-7-cleaved
(Asp198), caspase-9-cleaved (Asp330), caspase-3-cleaved (Asp175),
ERK 1/2 (Thr202/Tyr204), pPKC-z (Thr410/403), Src (Tyr527), Stat1
(Tyr701), and Bax
1:1000 for 4E-BP1 (Thr37/46) and Bcl-xL
1:2000 for eIF4G (Ser1108)

Recombinant protective antigen (PA), lethal factor (LF), and edema factor (EF) were obtained from List Biological Laboratories (Campbell, Calif.).

Ciprofloxacin, 1B-MECA, and Cl-IB-MECA were obtained from Sigma (St Louis, Mo.).

YVAD [that is: acetyl-tyrosyl-valyl-alanyl-aspartyl-chloromethylketon] was obtained from Bachem Bioscience (King of Prussia, Pa.).

Anthrax toxins were obtained from List Biological Labs (Campbell, Calif.).

### Example 2

### Challenge of Lung Epithelial Cells with Spores

HSAECs were grown in Ham's F12 media supplemented with non-essential amino acids, pyruvate, β-mercaptoethanol, and 10% FCS.

Confluent HSAECs (seeded at 10⁶/well in 12-well plates) were starved in the same media as above but containing 1% FCS for 16 hours and then challenged with spores. As shown in the figures, as described below, cells were cultured for up to 12 hours after challenge. Supernatants were removed, and cells were lysed and immediately boiled for 10 min in 100 μl of a 1:1 mixture of T-PER Reagent (Pierce, Rockford, Ill.) and 2× Tris-glycine SDS sample buffer (Novex/Invitrogen) in presence of 2.5% β-mercaptoethanol and protease inhibitors. Lysed samples were stored at −80° C. prior to use.

### Example 3

### Slide Printing and Staining

Nine nl of each sample were arrayed by a direct contact pin arrayer (Aushon Biosystems, Burlington, Mass.) onto nitrocellulose slides (Whatman, Mass.). Samples were printed in duplicate and in five-point 1:2 dilution curves to ensure a linear detection range for the antibody concentrations used. Slides were kept at −20° C. before analysis with antibodies.

To estimate total protein content, selected slides were stained with Sypro Ruby Protein Blot Stain (Molecular Probes, Eugene, Oreg.) and visualized on a Fluorchemk imaging system (Alpha Innotech, San Leandro, Calif.).

Antibodies were pre-validated for specificity by western blotting and peptide competition.

Slides were stained with pre-validated specific antibodies on an automated slide stainer (Dako, Carpinteria, Calif.) using a biotin-linked peroxidase catalyzed signal amplification. The arrayed slides were placed into 1× Re-Blot solution (Chemicon, Temecula, Calif.) for 15 min, washed two times for 5 min each in PBS, placed into I-Block solution (Applied Biosystems, Foster City, Calif.) in PBS/0.1% Tween-20 for at least 2 hours and then immunostained using the automatic slide stainer (Autostainer, Dako Cytomation, Carpinteria, Calif.) using manufacturer-supplied reagents. Briefly, the slides were incubated for 5 min with hydrogen peroxide, rinsed with high-salt Tris-buffered saline (CSA Buffer, Dako) supplemented with 0.1% Tween-20, blocked with avidin block solution for 10 min, rinsed with CSA buffer, and then incubated with biotin block solution for 10 min. After another CSA buffer rinse, a 5 min incubation with Protein Block solution was followed by air-drying.

The slides then were incubated with either a specific primary antibody diluted in Dako Antibody Diluent or, as a control, with only DAKO Antibody Diluent for 30 min.

The slides were then washed with CSA buffer and incubated with a secondary biotinylated goat anti-rabbit IgG H+L antibody (1:5000) (Vector Labs, Burlingame, Calif.) for 15 min.

For amplification purposes, the slides were washed with CSA buffer and incubated with streptavidin-horseradish peroxidase (HRP) for 15 min, followed by a CSA buffer rinse.

Slides were then incubated in diaminobenzidine (DAB) chromogen diluted in Dako DAB diluent for 5 min, washed in deionized water and imaged using a UMAX PowerLook III scanner (UMAX, Dallas, Tex.) at 600 dpi.

The images were analyzed with software AlphaEase FC (Alpha Innotech, San Leandro, Calif.). For each antibody, average pixel intensity value for the negative control (staining with only second antibody) was subtracted from the average pixel intensity and the resulting quantity then was divided by the corresponding value of the Sypro-stained total protein slides assays described above.

Two separate independent experiments were performed, and the averages of the measurements were analyzed. Positive and negative controls, consisting of A431 cells, respectively, treated and not treated with EGF, were printed on every slide array and served as reference standards for antibody performance.

Western blots were used to independently confirm the reverse phase protein microarray data. 20 μl of cell lysates were used for Western blots, which were stained with 1:1000-diluted primary antibody and 1:7500-diluted secondary antibody. Primary and secondary antibodies were the same as used for the reverse phase protein microarray. Reverse-phase protein microarray and Western blot data are presented as the average of two independent experiments. For reverse-phase protein microarray assays each sample was printed in duplicate.

### Example 4

### Animal Experiments

DBA/2 male mice (Jackson Labs), 6 to 8 weeks old, received food and water ad libitum, and were challenged with anthrax spores (1×10⁷ spores, i.p.) on day 0.

Survival of animals was monitored daily for 15 days.

Ciprofloxacin (Sigma, St Louis, Mo.) treatment (50 mg/kg, once daily, i.p.) was initiated at day +1, simultaneously with administration of inhibitors, and continued for 10 days. Two doses (2.5 mg/kg and 12.5 mg/kg) of YVAD (acetyl-tyrosyl-valyl-alanyl-aspartyl-chloromethylketone from Bachem Bioscience, Pa.) and 3 doses (0.05, 0.15 and 0.3 mg/kg) of Cl-IB-MECA (1-[2-Chloro-6-[[(3-iodophenyl)methyl]amino]-9H-purin-9-yl]-1-deoxy-N-methyl-β-D-ribofuranuronamide from Sigma, St Louis, Mo.) were administered. Animals received YVAD on days +1 to +4, and Cl-IB-MECA on days +1 to +10 once daily, s.c.

### Example 5

### Matched Isogenic Pathogenic and Non-Pathogenic Strains

To more specifically identify cell signaling pathways that are causally important or related to the infectious process directly, signal pathway profiling was carried out using matched isogenic strains of B. anthracis: pathogenic strain Sterne (pXO1⁺, pXO2⁻) and non-pathogenic anthrax strain (delta-Sterne (pXO1⁻, pXO2⁻). Pathogenic effects of B. anthracis were determined by exposing lung epithelial cells to each of the strains, separately, and monitoring subsequent changes in cell physiology, as described, for instance, in other examples herein.

The use of these matched, isogenic pathogenic and non-pathogenic strains provides a means to identify pathogenic host responses to anthrax virulence factors encoded by the pXO1 plasmid. These strains and their use is an important advance over previous studies of B. anthracis infections that utilized only virulent strains or toxins without matched isogenic controls, such as those provided by the strains discussed above.^{21,22,23,24,25}

The matched strains also provide the ability to study the late bacteremic stages of infection, when anthrax-encoded secreted toxins along with other pathogenic factors are thought to be involved in the damage to vital organs with high epithelial content, such as lung, liver, spleen, and kidney. Results from in vitro studies of exposure of primary lung epithelial cells to matched pathogenic and non-pathogenic strains, accordingly, should provide valuable guidance for animal studies and for prophylactic and therapeutic intervention to prevent, mitigate, or cure anthrax, as provided further in other examples herein.

### Example 6

### Phosphorylation of Signaling Proteins

The dynamics of cell signaling phosphorylation in the Human Small Airway Epithelial Cells (HSAECs) after exposure to anthrax spores was determined using an array of 43 different antibodies, as described in Example 1. The specificity of each antibody (set forth above, in Example 1) had been validated in previous studies. See Espina et al., J. Immuno. Meth. 290(1-2): 121-133 (2004) and in references therein, all of which are herein incorporated by reference in their entireties, particularly in parts pertinent to the foregoing antibodies and their validation and the like. The panel was selected based on the ability of the antibodies to broadly monitor the molecular networks involved in host response pathways most likely to be affected by bacterial exposure: namely survival, apoptosis, inflammation, growth, differentiation, and immune responses.

### Example 7

### Signaling Protein Phosphorylation in Host Pathogenic Responses

Changes in phosphorylation of host cell signaling proteins were determined by comparing phosphorylation of signaling proteins in HSAECs exposed either to pathogenic B. anthracis strain Sterne (pXO1⁺, pXO2⁻) or non-pathogenic B. anthracis strain Sterne (pXO1⁻, pXO2⁻) (“delta Sterne”). Phosphorylation of the proteins was determined in all cases using antibody panels as described above.

Two independent experiments were performed and the results were averaged. 24 of the 43 signaling endpoints were statistically changed upon exposure to either strain. 6 of those exhibiting the most significant changes were further tested and verified by western blot analysis.

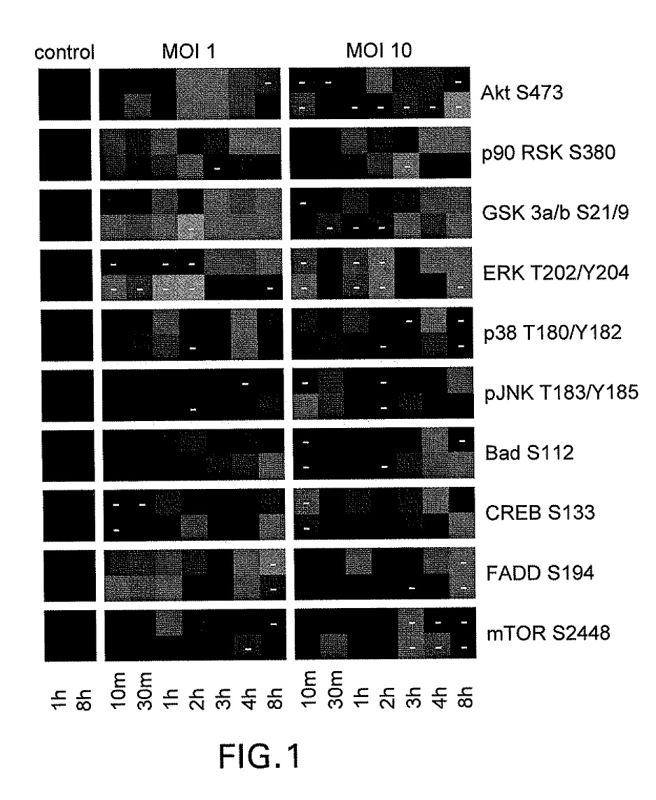

Among these the most prominent difference was that phosphorylation of proteins of the pro-survival signaling pathway was considerably greater in cells exposed to the pathogenic strain than it was in cells exposed to the non-pathogenic strain. This was particularly true for phosphorylation of mitogen-activated protein kinases (MAPKKs) ERK1/2 (p44/42 MAPK), their downstream target p90 RSK, other members of the MAPK family, such as the stress-activated kinases p38 and JNK, and the global regulators of survival pathways—the serine/threonine kinases AKT1/2. Representative results in this regard are depicted graphically in FIG. 1.

Increased phosphorylation of ERK and AKT kinases is generally accepted as serving a protective role, directed to the elimination of non-pathogenic bacteria.^{26,27,28} AKT is a pluripotent mediator of a number of cellular processes. It provides a crucial link between P13 kinase and anti-apoptotic mediators, and is one of the most important mediators of cell survival.²⁹ Down-regulation of ERK and AKT phosphorylation in the epithelium upon exposure to the pathogenic strain compared to the non-pathogenic strain pointes to a pathogenic mechanism leading to suppression of epithelial survival and abrogation of the protective functions of epithelial cells. MAPKKs are known to be specific targets of lethal toxin (LeTx) proteolytic activity^{30,31} and they are implicated in the induction of apoptosis by LeTx in macrophages and epithelial cells.^{32,33} The effect of anthrax exposure on AKT phosphorylation in target host cells, however, is a novel observation. And, since AKT regulates glycogen synthesis by phosphorylation of glycogen synthase kinase 3 (GSK3), AKT inactivation may play an important role in the abnormal glucose levels observed both in animals exposed experimentally to anthrax and in human patients suffering from anthrax infection.

Also notable is the link between the AKT activity and the adenylate cyclase activity of edema toxin (EdTx). Cyclic AMP (cAMP) and its effector cAMP-dependent protein kinase (PICA) together are an integral component of many intracellular signaling pathways.

In many cell types an increase in the level of cellular cAMP inhibits cell growth and inhibits AKT signaling, by blocking the coupling of AKT with its upstream regulators.^{34,35} As a result, EdTx adenylate cyclase activity may mediate the decreased AKT phosphorylation engendered in cells exposed to toxigenic B. anthracia.

### Example 8

### AKT Phosphorylation in HSAECs in Response to EdTx

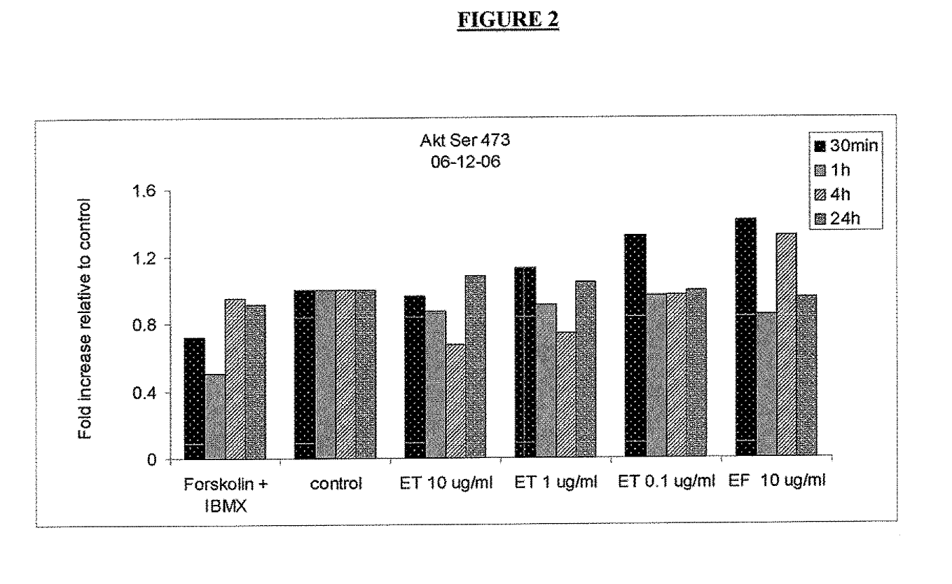

In view of the foregoing results, and the importance of AKT to survival pathways, the effect of EdTx on AKT phosphorylation was studied in greater detail. Representative results depicted in. FIG. 2 show that that AKT phosphorylation in HSAECs exposed to different concentrations of EdTx first increases, then decreases, over a period of at least 8 hours and, finally, returns almost to normal within 24 hours. LeTx, at the same time, does not affect AKT phosphorylation in HSAECs (data not shown). This pattern of AKT phosphorylation matches the cAMP-mediated physiological response of experimental animals to prolonged intravenous infusion of epinephrine which leads to the same type of vascular collapse that has long been recognized as a major aspect of the terminal phase of anthrax infection.^{36,37}

While no in vitro system can model with complete fidelity the entire complexity of host-pathogen infectious processes that occur in intact organisms, results obtained using HSAECs, among others, are nonetheless reasonably predictive of the results to be expected in vivo and remain particularly valuable for testing therapeutic approaches that target the host cell response. Taken together, the results indicate that pharmacologically correcting the altered host cell intracellular signaling, could affect the lethal outcome in anthrax-challenged animals.

### Example 9

### Protective Effect of YVAD in an Animal Model, Alone and when Used in Combination with Another Agent

According to Smith et al., antibiotics alone cannot treat anthrax after a certain time of “no return” when toxemia becomes a predominant factor. In certain embodiments of the invention several agents are used in combination, with one another and/or with an antibiotic. In the case of anthrax infection, for instance, each of the illustrative agents individually can correct one or more signaling abnormalities caused by either or both LeTx and EdTx, and they can be used alone or in combination with one another and/or in combination with an antibiotic, such as ciprofloxacin, which targets the bacterial proliferation.

Thus, by way of example, it has been shown that apoptosis is induced by LeTx in cultured macrophages and in the livers of anthrax-challenged mice. It also has been shown that the general caspase inhibitor, z-Val-Ala-Asp(OMe)-fluoromethylketone (“z-VAD”) and the specific caspase-¼ inhibitor, acetyl-tyrosyl-valyl-alanyl-aspartyl-chloromethylketone (“YVAD”), each has a protective anti-apoptopic effect in both of these models.³⁸

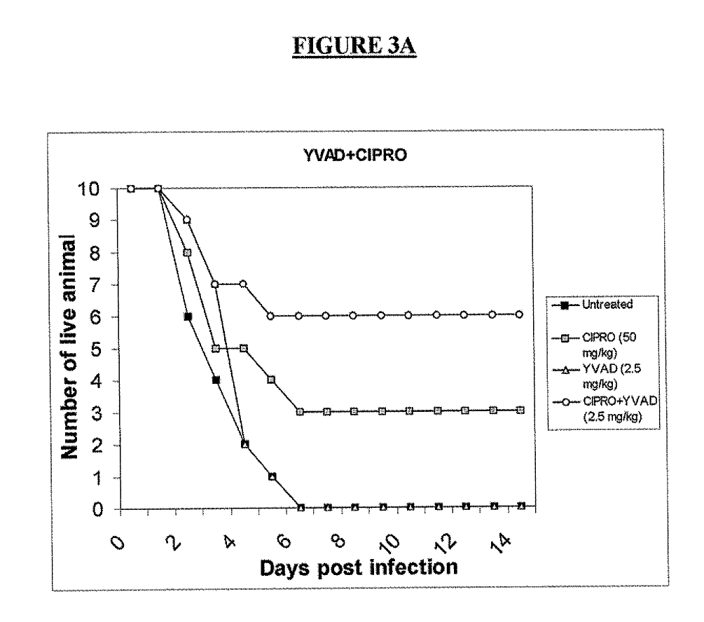

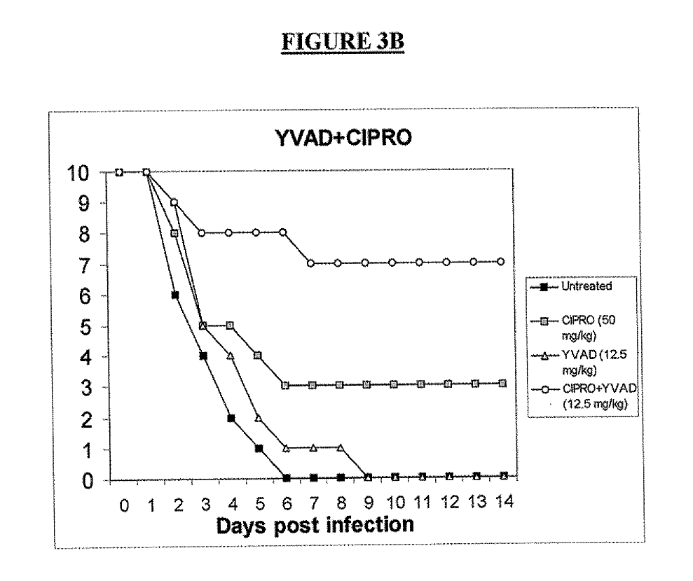

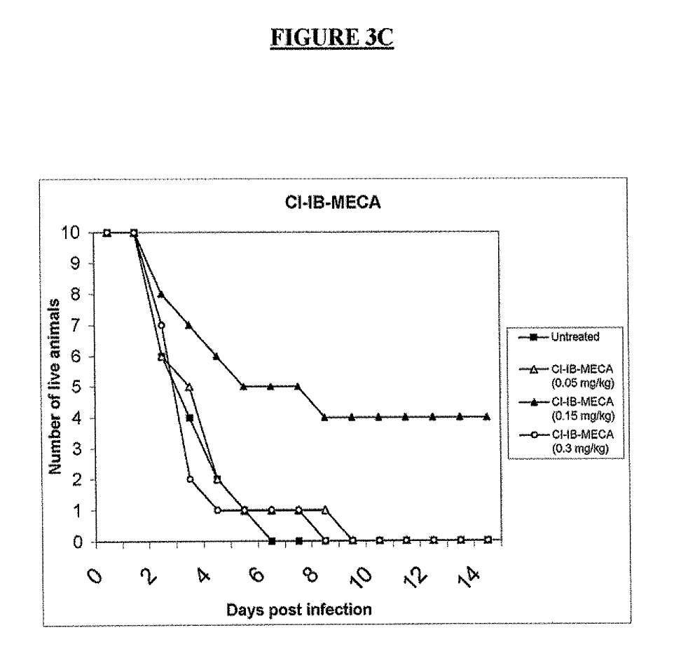

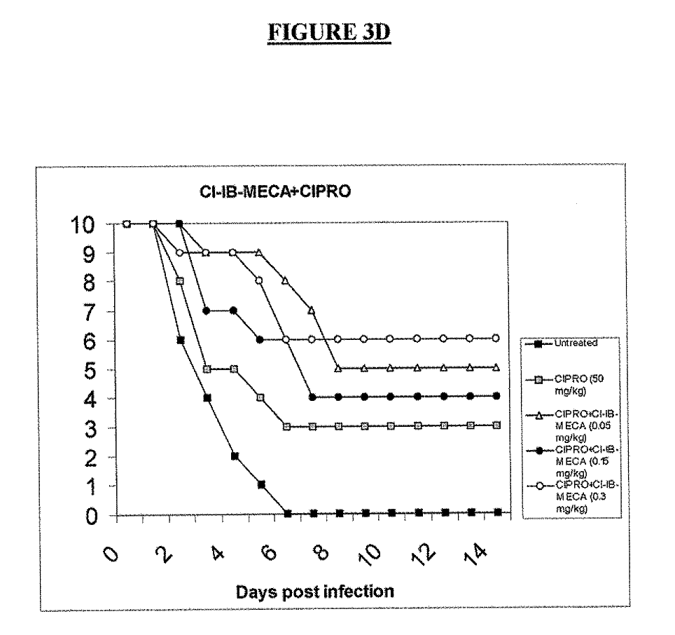

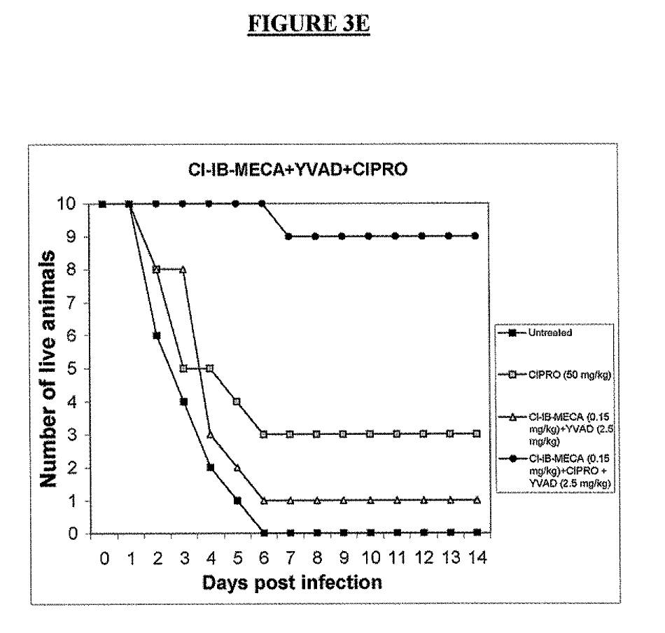

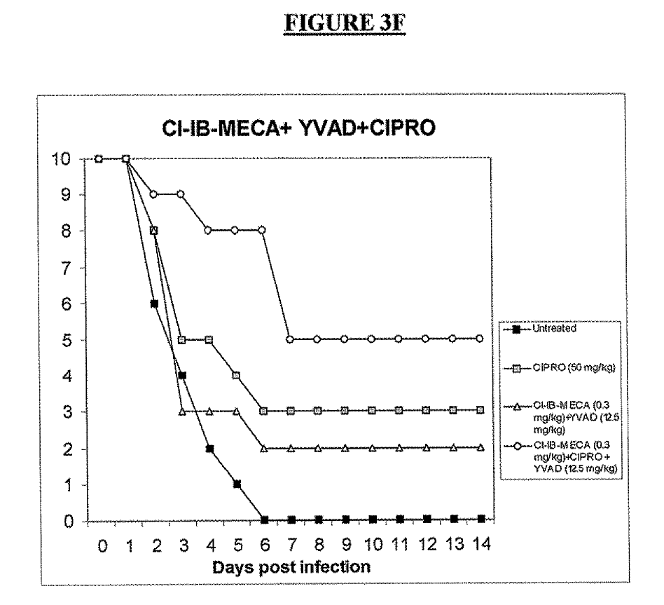

As depicted in FIG. 3, only four doses of YVAD in combination with ciprofloxacin, administered on days +1 through +4 post infection protect up to 70% of animals over the course of the experiment from days +1 through +10. Under the same conditions, with the same delay in beginning treatment until day +1 post-exposure (which better models likely clinical circumstances), administration of ciprofloxacin by itself protected only 30% of animals.

### Example 10

### Protective Effect of Cl-IB-MECA in an Animal Model Alone and when Used in Combination with Other Agents

Host AKT pathway responses to anthrax exposure and infection provide an example of a host cell response that may be targeted for protection and therapy, as shown in this example. Since host cell AKT phosphorylation is decreased as a result of exposure to pathogenic B. anthracis, beneficial effects thus may be obtained by counteracting this effect, and restoring AKT phosphorylation to its normal levels. The results in this example show that pharmacologically altering cAMP-mediated host cell AKT signaling is protective against the pathological effects of anthrax exposure and infection.

AKT activity is a function of its phosphorylation. Phosphorylation of AKT in part, depends on cAMP levels; although, the effect is indirect. In many cells, down regulation of cellular cAMP decreases phosphorylation AKT, and that of other regulatory signaling kinases such as ERK1/2 and GSK3β (S9).^{39,40} (In other cells, however, the effect is just the opposite.) Cellular cAMP levels are influenced and often, in part, regulated directly, by A3ARs. Accordingly, phosphorylation of AKT can be increased, and its activity restored in cells exposed to anthrax by stimulating A3ARs to decrease cellular cAMP levels. Increased AKT activity, for the reasons set forth above, protects cells against the deleterious results of anthrax infection. In addition, the stimulation of A3ARs, by itself, may provide prophylactic and/or therapeutic effects against anthrax. In particular, modulation of A3AR activities is known to be cardioprotective during hypoxia,⁴¹ to inhibit apoptosis, to protect against endotoxemia⁴² and colitis,⁴³ and to decrease renal and hepatic injury, and mortality in sepsis.⁴⁴

The results in this example show that pharmacological stimulation of adenosine A3 receptors (A3ARs), which leads to AKT phosphorylation and activation protects animals from developing anthrax after exposure to B. anthracis. The results show, furthermore, that the protective and therapeutic effect of the treatment is increased when the agents for stimulating the A3ARs are used in combination with other therapeutic agents, such as antibiotics.

By way of illustration in this regard, the results in this example show that two A3AR agonists IB-MECA (N⁶-(3-iodobenzyl) adenosine-5′-N-methyluronamide) and Cl-IB-MECA, its Cl-substituted derivative, protect mice against post-exposure anthrax.

As shown in FIG. 3C treatment with Cl-IB-MECA, at the optimal dose of 0.15 mg/kg, resulted in 40% protection of exposed animals, even without co-administration of the antibiotic, ciprofloxacin. This was a remarkable result. Moreover, administration of Cl-IB-MECA in combination with ciprofloxacin was more effective than administration of ciprofloxacin, at a variety of doses, and the difference was statistically significant for the results obtained by administration of 0.3 mg/kg of Cl-IB-MECA (p<0.05).

As shown in FIGS. 3E and 3F, even better results were obtained using triple combinations of YVAD, Cl-IB-MECA, and ciprofloxacin, which target generation of IL-1β, A3AR activity, and bacterial growth, respectively. As seen in the figures, the triple combination was synergistic and protected up to 90% of exposed animals, Combinations of YVAD and Cl-IB-MECA without ciprofloxacin were only marginally more protective when used together than when either one or the other was used by itself.

These results indicate that the down-regulation of the ERK and AKT survival axis as observed in host lung epithelial cells is not a simple correlative finding, but is casually important in disease pathogenesis and overall anthrax-induced mortality. These findings also indicate that an optimal combination post-exposure anthrax therapy based entirely on the modulation of the host response to infection could be highly effective, and highly synergistic with the current state-of-care antibiotic, ciprofloxacin.

### REFERENCES

The following references are each individually incorporated herein by reference in their entirety, particularly in parts pertinent to the invention herein disclosed, particularly as to the subject matter of their specific citation described and indicated where cited herein above.
¹Holty J E, Bravata D M, Liu H, Olshen R A, McDonald K M, Owens D K. Systematic review: a century of inhalational anthrax cases from 1900 to 2005. Ann Intern Med. 2006 Feb. 21; 144(4):270-80.
²Pomerantsev A P, Shishkova N A, Marinin L I (1992). Comparison of therapeutic effects of antibiotics of the tetracycline group in the treatment of anthrax caused by a strain inheriting tet-gene of plasmid pBC16. [Article in Russian] Antibiot Khimioter. 37(4):31-34.
³Pomerantsev A P, Staritsin N A, Mockov Yu V, Marinin L I (1997). Expression of cereolysine AB genes in Bacillus anthracis vaccine strain ensures protection against experimental hemolytic anthrax infection. Vaccine. 15(17-18):1846-1850.
⁴Inglesby T V, O'Toole T, Henderson D A, Bartlett J G, Ascher M S, Eitzen E, Friedlander A M, Gerberding J, Hauer J, Hughes J, McDade J, Osterholm M T, Parker G, Perl T M, Russell P K, Tonat K. Anthrax as a biological weapon, 2002: updated recommendations for management. JAMA. 2002 May 1; 287(17):2236-52.
⁵Moayeri M, Leppla S H. The roles of anthrax toxin in pathogenesis. Curr Opin Microbiol. 2004 February; 7(1):19-24.
⁶Shoop W L, Xiong Y, Wiltsie J, Woods A, Guo J, Pivnichny J V, Felcetto Bansal A, Cummings R T, Cunningham B R, Friedlander A M, Douglas C M, Patel S B, Wisniewski D, Scapin G, Salowe S P, Zaller D M, Chapman K T, Scolnick E M, Schmatz D M, Bartizal K, MacCoss M, Hermes J D. Anthrax lethal factor inhibition. Proc Natl Acad Sci USA. 2005 May 31; 102(22):7958-63.
⁷Popov S G, Popova T O, Hopkins S, Weinstein R S, MacAfee R, Fryxell K J, Chandhoke V, Bailey C, Alibek K. Effective antiprotease-antibiotic treatment of experimental anthrax. BMC Infect Dis. 2005 Apr. 8; 5(1):25.
⁸Vancurik J. Causes of the failure of antibiotic prophylaxis of inhalation anthrax and clearance of the spores from the lungs. Folia Microbial (Praha). 1966; 11(6): 459-64.
⁹Wulfkuhle J D, Edmiston K H, Liotta L A, Petricoin E F 3rd. Technology Insight: pharmacoproteomics for cancer—promises of patient-tailored medicine using protein microarrays. Nat Clin Pract Oncol. 2006 May; 3(5):256-68.
¹⁰Tucker A E, Salles I I, Voth D E, Ortiz-Leduc W, Wang H, Dozmorov I, Centola M, Ballard J D. Decreased glycogen synthase kinase 3-beta levels and related physiological changes in Bacillus anthracis lethal toxin-treated macrophages. Cell Microbiol. 2003 August; 5(8):523-32.
Bergman N H, Passalacqua K D, Gaspard R, Shetron-Rama L M, Quackenbush J, Hanna P C. Murine macrophage transcriptional responses to Bacillus anthracis infection and intoxication. Infect Immun. 2005 February; 73(2):1069-80.
¹²Corner J E, Galindo C L, Zhang F, Wenglikowski A M, Bush K L, Garner H R, Peterson J W, Chopra A K. Murine macrophage transcriptional and functional responses to Bacillus anthracis edema toxin. Microb Pathog. 2006 Jul. 15; [Epub ahead of print].
¹³Corner J E, Chopra A K, Peterson J W, Konig R. Direct inhibition of T-lymphocyte activation by anthrax toxins in vivo. Infect Immun. 2005 December; 73(12):8275-81.
¹⁴corner J E, Galindo C L, Chopra A K, Peterson J W. GeneChip analyses of global transcriptional responses of murine macrophages to the lethal toxin of Bacillus anthracis. Infect Immun. 2005 March; 73(3):1879-85.
¹⁵Corner J E, Chopra A K, Peterson J W, Konig R Infect Immun. Direct inhibition of T-lymphocyte activation by anthrax toxins in vivo. 2005 December; 73(12):8275-81.
¹⁶Eckert N, Bonventre P. In vivo effects of Bacillus anthracis culture filtrates.
¹⁷Stein M W, Logan G F Jr. Mechanism of action of the toxin of Bacillus anthracis. I. Effect in vivo on some blood serum components. J Bacteriol. 1960 July; 80:77-85.
¹⁸Remmele N S, Klein F, Vick J A, Walker J S, Mahlandt B G, Lincoln R E. Anthrax toxin: primary site of action. J Infect Dis. 1968 February; 118(1):104-13.
¹⁹van Nieuw Amerongen G P, van Hinsbergh V W. Targets for pharmacological intervention of endothelial hyperpermeability and barrier function. Vascul Pharmacol. 2002 November; 39(4-5):257-72.
²⁰Zhu W Z, Zheng M, Koch W J, Lefkowitz R J, Kobilka B K, Xiao R P. Dual modulation of cell survival and cell death by beta(2)-adrenergic signaling in adult mouse cardiac myocytes. Proc Natl Acad Sci USA. 2001 Feb. 13; 98(4):1607-12.
²¹Tucker A E, Salles I I, Voth D E, Ortiz-Leduc W, Wang H, Dozmorov I, Centola M, Ballard J D. Decreased glycogen synthase kinase 3-beta levels and related physiological changes in Bacillus anthracis lethal toxin-treated macrophages. Cell Microbiol. 2003 August; 5(8):523-32.
²²Bergman N H, Passalacqua K D, Gaspard R, Shetron-Rama L M, Quackenbush J, Hanna P C. Murine macrophage transcriptional responses to Bacillus anthracis infection and intoxication. Infect Immun. 2005 February; 73(2):1069-80.
²³Corner J E, Galindo C L, Zhang F, Wenglikowski A M, Bush K L, Gamer H R, Peterson J W, Chopra A K. Murine macrophage transcriptional and functional responses to Bacillus anthracis edema toxin. Microb Pathog. 2006 Jul. 15; [Epub ahead of print].
²⁴Corner J E, Chopra A K, Peterson J W, Konig R. Direct inhibition of T-lymphocyte activation by anthrax toxins in vivo. Infect Immun. 2005 December; 73(12):8275-81.
²⁵corner J E, Galindo C L, Chopra A K, Peterson J W. GeneChip analyses of global transcriptional responses of murine macrophages to the lethal toxin of Bacillus anthracis. Infect Immun. 2005 March; 73(3):1879-85.
²⁶Brazil D P, Park J, Hemmings B A. Getting in on the Akt Cell. 2002 Nov. 1; 111(3):293-303.
²⁷Datta S R, Brunet A, Greenberg M E. Cellular survival: a play in three Akts. Genes Dev. 1999 Nov. 15; 13(22):2905-27.
²⁸Wesche D E, Lomas-Neira J L, Perl M, Chung C S, Ayala A. Leukocyte apoptosis and its significance in sepsis and shock. J Leukoc Biol. 2005 August; 78(2):325-37.
²⁹Franke T F, Hornik C P, Segev L, Shostak G A, Sugimoto C. PI3K/Akt and apoptosis: size matters. Oncogene. 2003 Dec. 8; 22(56):8983-98.
³⁰Duesbery N S, Vande Woude G F. Anthrax lethal factor causes proteolytic inactivation of mitogen-activated protein kinase kinase. J Appl Microbiol. 1999 August; 87(2):289-93.
³¹Pellizzari R, Guidi-Rontani C, Vitale G, Mock M, Montecucco C. Lethal factor of Bacillus anthracis cleaves the N-terminus of MAPKKs: analysis of the intracellular consequences in macrophages. Int J Med Microbiol. 2000 October; 290(4-5):421-7.
³²Popov S G, Villasmil R, Bernardi J, Grene E, Cardwell J, Wu A, Alibek D, Bailey C, Alibek K. Lethal toxin of Bacillus anthracis causes apoptosis of macrophages. Biochem Biophys Res Commun. 2002 Apr. 26; 293(1):349-55.
³³Park J M, Greten F R, Li Z W, Karin M. Science. 2002 Sep. 20; 297(5589):2048-51. Macrophage apoptosis by anthrax lethal factor through p38 MAP kinase inhibition.
³⁴Wang L, Liu F, Adamo M L. Cyclic AMP inhibits extracellular signal-regulated kinase and phosphatidylinositol 3-kinase/Akt pathways by inhibiting Rapl. J Biol Chem. 2001 Oct. 5; 276(40):37242-9.
³⁵Liu L, Xie Y, Lou L. Cyclic AMP inhibition of proliferation of hepatocellular carcinoma cells is mediated by Akt. Cancer Biol Ther. 2005 November; 4(11):1240-7.
³⁶Singh K, Xiao L, Remondino A, Sawyer D B, Colucci W S. Adrenergic regulation of cardiac myocyte apoptosis. J Cell Physiol. 2001 December; 189(3):257-65.
³⁷Williams R P, Hill H R, Hawkins D, Chao K-C, Neuenschwander J, Lipscomb H S. Epinephrinelike activity of culture filtrate from Bacillus anthracis. Fed Proc. 1967; 26(5):1545-48.
³⁸Popov S G, Popova T G, Grene E, Klotz F, Cardwell J, Bradburne C, Jama Y, Maland M, Wells J, Nalca A, Voss T, Bailey C, Alibek K. Systemic cytokine response in murine anthrax. Cell Microbiol. 2004 March; 6(3):225-33.
³⁹Park S S, Zhao H, Jang Y, Mueller R A, Xu Z. N6-(3-Iodobenzyl)-adenosine-5′-N-methylcarboxamide confers cardioprotection at reperfusion by inhibiting mitochondrial permeability transition pore opening via glycogen synthase kinase 3beta. J Pharmacol Exp Ther. 2006 July; 318(1):124-31.
⁴⁰Matot I, Weiniger C F, Zeira E, Galun E, Joshi B V, Jacobson K A. A3 adenosine receptors and mitogen-activated protein kinases in lung injury following in vivo reperfusion. Crit Care. 2006 Apr. 19; 10(2):R65 [Epub ahead of print].
⁴¹Panjehpour M, Karami-Tehrani F. An adenosine analog (IB-MECA) inhibits anchorage-dependent cell growth of various human breast cancer cell lines. Int J Biochem Cell Biol. 2004 August; 36(8):1502-9.
⁴²Hasko G, Nemeth Z H, Vizi E S, Salzman A L and Szabo C. An agonist of aden receptors decreases interleukin-12 and interferon-gamma production and prevents lethality in endotoxemic mice. Eur J Pharmacol. 1998; 358:261-68.
⁴³Mabley J, Soriano F, Pacher P, Hasko G, Marton A, Wallace R, Salzman A and Szabo C. The adenosine A3 receptor agonist, N6-(3-iodobenzyl)-adenosine-5′-N-methyluronamide, is protective in two murine models of colitis. Eur J Pharmacol. 2003; 466: 323-29.
⁴⁴Lee H, Kim M, Joo J D, Gallos G, Chen J F, Emala C W. A3 Adenosine Receptor Activation Decreases Mortality, Renal and Hepatic Injury in Murine Septic Peritonitis. Am J Physiol Regul Integr Comp Physiol. 2006 May 25; [Epub ahead of print].

1.-7. (canceled)

8. A method for preventing and/or treating anthrax infection, comprising administering to a subject at risk for or suffering from anthrax infection a first agent that inhibits the activity of caspase ¼ and a second agent that increases the phosphorylation of AKT, wherein said first and said second agents each are administered in an amount and by a route effective for preventing and/or treating said anthrax infection in combination with one another.

9. A method according to claim 8, wherein the agent that increases the phosphorylation of AKT is an agonist of an adenosine A3 receptor.

10. A method according to claim 9, wherein the agent that increases the phosphorylation of AKT is IB-MECA or Cl-IB-MECA.

11. A method according to claim 8, wherein the agent that inhibits the activity of caspase ¼ is YVAD.

12. A method according to claim 11, wherein the agent that increases the phosphorylation of AKT is an agonist of an adenosine A3 receptor.

13. A method according to claim 12, wherein the agent that increases the phosphorylation of AKT is IB-MECA or Cl-IB-MECA.

14. A method according to claim 8, further comprising administering an antibiotic to said subject, wherein said antibiotic is administered in an amount and by a route effective for preventing and/or treating said anthrax infection in combination with said agents.

15. A method according to claim 14, wherein the antibiotic is ciprofloxacin.

16.-20. (canceled)

21. A pharmaceutically acceptable composition comprising a first agent that decreases the activity of caspase ¼, a second agent that increases the phosphorylation of AKT, and an antibiotic.

22. A pharmaceutically acceptable composition according to claim 21, wherein the first agent is YVAD.

23. A pharmaceutically acceptable composition according to claim 21, wherein said second agent is an agonist of an adenosine A3 receptor.

24. A pharmaceutically acceptable composition according to claim 23, wherein the second agent is IB-MECA or Cl-IB-MECA.

25. A pharmaceutically acceptable composition according to claim 22, wherein the second agent is an agonist of an adenosine A3 receptor.

26. A pharmaceutically acceptable composition according to claim 25, wherein the second agent is IB-MECA or Cl-IB-MECA.

27. A pharmaceutically acceptable composition according to claim 16, wherein the antibiotic is ciprofloxacin.

28. A pharmaceutically acceptable composition according to claim 16, wherein the composition is effective for preventing and/or treating anthrax infection.

29.-33. (canceled)

34. A kit, comprising in one or more containers a pharmaceutically acceptable composition comprising a first agent that decreases the activity of caspase ¼, a second agent that increases the phosphorylation of AKT, an antibiotic, and instructions for the pharmaceutical use thereof.

35. A kit according to claim 34, wherein said first agent is YVAD.

36. A kit according to claim 34, wherein said second agent is an agonist of an adenosine A3 receptor.

37. A kit according to claim 36, wherein said second agent is IB-MECA or Cl-IB-MECA.

38. A kit according to claim 35, wherein said second agent is an agonist of an adenosine A3 receptor.

39. A kit according to claim 38, wherein said second agent is IB-MECA or Cl-IB-MECA.

40. A kit according to claim 29, wherein the antibiotic is ciprofloxacin.

41.-44. (canceled)
